(19) United States
(12) Patent Application Publication
(10) Pub. No.: US 2026/0315388 A1
(43) Pub. Date: Oct. 15, 2026

(54) COMPOSITIONS AND TREATMENT METHODS FOR PROMOTING BRAIN HEALTH

(71) Applicant: GEROSCIENCES, LLC, Lighthouse Point, FL (US)

(72) Inventors: Gregory Horn, Lighthouse Point, FL (US); Rudolf Leonardus Lodewijk Smeets, Venlo (NL); Sreeraj Gopi, Kerala (IN); Guru Ramanathan, Gibsonia, PA (US)

(21) Appl. No.: 19/175,273

(22) Filed: Apr. 10, 2025

(51) Int. Cl. A61K 9/1271; A61K 9/00; A61K 31/26; A61K 31/352; A61K 31/7048; A61K 47/12; A61K 47/26

(52) U.S. Cl. CPC A61K 9/1271; A61K 9/0053; A61K 31/26; A61K 31/352; A61K 31/7048; A61K 47/12; A61K 47/26

(57) ABSTRACT

A method for preparing a surface modified integrated lipid encapsulated structure for a formulation for treating and managing cognitive function and impairment comprising the steps of preparing a crosslinking biopolymer for surface modification; preparing a lipid based vehicle for encapsulation of a bioactive; and modifying the surface of the lipid vesicle, and its applications for treating and managing cognitive function and impairment.

### BACKGROUND OF THE INVENTION

The present invention relates to the promotion of brain health, and in particular a composition, and combination of compositions, for use in treatment and prevention of memory deterioration diseases.

The pharmaceutical industry has developed a strong focus to find treatments for several of the different disorders affecting brain health, however with only limited success. There are still no cures available for diseases like Alzheimer's and Parkinson's. There is also no effective strategy to repair brain damage caused by injuries or infections.

Brain health is the state of brain functioning across cognitive, sensory, social-emotional, behavioral, and motor domains. The goal of maintaining or improving brain health is allowing a person to realize their full potential over the life course, irrespective of the presence or absence of disorders (WHO Health Topics Brain Health, 2022).

Optimizing brain health improves both mental and physical health, leading to a positive social and economic impact for the individual and society. As life span increases, global momentum is increasing to address brain health. The World Health Organization (“WHO”) acknowledged the importance by launching a special action plan for the coming decade (Intersectional global action plan on epilepsy and other neurological disorders 2022-2031) (Optimizing brain health across the life course, 2022). The focus on brain health is justified knowing that one in three people globally will develop a neurological disorder at some point in their lifetime. Neurological disorders account for the second leading cause of death and the leading cause of disability adjusted life years (“DALY”s). In the US alone, it is estimated that the financial costs of neurological disorders are close to $800 billion (Gooch, Pracht, & Borenstein, 2017).

As seen in Table 1 below, a list of disorders which affect brain health is shown.

TABLE I
Disorders affecting brain health.

| | Neurological | | | | |
|---|---|---|---|---|---|
| Common | complications | | | Neuro- | |
| Neurological | due to | | | developmental | Congenital |
| conditions | infections | Cancer | Injuries | disorders | disorders |
| Dementia | COVID-19 | CNS | Traumatic | ADHD | Congenital |
| | | tumors | brain | | birth defects |
| | | | injury | | |
| Epilepsy | HIV | | Spinal | Autism | Down |
| | | | cord injury | | syndrome |
| Stroke | Malaria | | | Cerebral Palsy | Neural tube |
| | | | | | defects |
| Parkinson | Encephalitis | | | Fetal alcohol | Other genetic |
| | | | | syndrome | defects |
| Multiple | Meningitis | | | | |
| sclerosis | | | | | |
| Headache | Zika | | | | |
| disorders | | | | | |
| Motor | Cysticercosis | | | | |
| Neuron | | | | | |
| disease | | | | | |

The pharmaceutical industry has developed a strong focus to find treatments for several of the different disorders affecting brain health, however with only limited benefits. There are still no cures available for diseases like Alzheimer's and Parkinson's. There is also no effective strategy to repair brain damage caused by injuries or infections.

The brain is a complex organ which complexifies finding effective therapeutic strategies based on 1 or 2 chemical substances. Even where a nutritional approach has been tried, an additional problem is the limited bioavailability of these nutritional ingredients in the brain after supplementation. An additional, but primary hurdle to brain health is the limited bioavailability of most nutritional ingredients in the brain after oral supplementation.

The brain weighs about 3 pounds in an average adult. The brain contains vessels for fluid transport (like blood) and nerves (Brain Anatomy and How the Brain Works, 2022). At a high level the brain can be divided into the cerebrum, brainstem, and cerebellum. The cerebrum is the large front part of the brain responsible for movement, thinking, problem-solving, emotions and learning. Parts of the cerebrum are the lobes (frontal lobe, parietal lobe, temporal lobe, and occipital lobe). The brainstem connects the cerebrum with the spinal cord and is responsible for movement and coordination but also chewing and focusing vision. The bottom of the brainstem is called the medulla. This is where the brain meets the spinal cord. The medulla regulates bodily activities including heart rhythm, blood flow and breathing. The medulla produces the reflexive activities like coughing and swallowing. The third part of the brain is called the cerebellum. It is also called little brain and is located at the back of the head. Besides being involved in balance and equilibrium it also plays a role in emotions and social behavior.

The brain utilizes four fluid types to maintain proper brain function by ensuring transport of nutrients to the brain tissue and by removing toxic waste products: 1) cerebrospinal fluid (“CSF”), found in the parenchyma, 2) interstitial fluid (ISF), 3) intracellular fluid, and 4) blood; citicoline (“CDP”) transported in vasculature. The blood is kept separate from the brain parenchyma and CSF by respectively the blood-brain and the blood-CSF barrier (Jessen, Finmann Munk, Lundgaard, & Nedergaard, 2015).

CSF is continuously produced and renewed around four times per 24 hours. The total volume of CSF is around 160 ml and is kept constant by removal of CSF through the peripheral lymphatic system. The volume is thought to be kept constant by hydrostatic and osmotic forces.

With respect to ingredient delivery, lipid-based encapsulation systems have garnered significant attention due to their outstanding properties such as high drug-loading capacity, lower toxicity, improved bioavailability and biocompatibility, stability in the gastrointestinal tract, controlled release, and simpler scale-up and validation processes. However, these systems face challenges like poor drug penetration, limited drug encapsulation, and inadequate targeting.

The current art includes formulations, such as those sold under the brand Souvenaid®, that are known in the art to treat early-stage Alzheimer's. Souvenaid® is an orally ingested combination of key nutrients, Specifically, it includes DHA, EPA, UMP, Choline,

Phospholipids, Folic acid, Vitamin B6, Vitamin B12, Vitamin C, Vitamin E, and Selenium. It is known that the GI system and the blood/brain barrier are major hurdles for the bioavailability of key nutrients in the brain. Therefore, Souvenaid® suffers from the disadvantage that it does not fully make its way to the brain, and when it does, it does not adequately solve the issues brought by Alzheimer's.

Accordingly, it is an object of the present invention to provide a formulation, method of manufacture and method of delivery which overcome the shortcomings of the prior art.

### SUMMARY OF THE INVENTION

To overcome the problem of bioavailability, the invention contains an improved combination of bioavailability-enhancing active ingredients, an improved method of manufacture, and an improved method of administration. The composition is provided in way that enhances the uptake of key individual ingredients for brain health by adding them together in an innovative surface modified lipid encapsulation structure. (“SMLE”). In one method of use, the bioavailability of the inventive formulation will be further improved by leveraging the glymphatic system activity during sleep.

The present invention utilizes surface modification, which results in controlled release, enhanced penetration efficiency, and targeted drug delivery using polymers, ligands, surfactants, and fatty acids.

The invention includes a composition most effective taken before sleep, a composition most effective taken during the day, and a treatment based upon taking both.

The inventive formula includes variations based upon 5 mg rutin and 5 mg lutein or more in a surface modified integrated lipid encapsulation.

### BRIEF DESCRIPTION OF THE DRAWINGS

The features and advantages of the present invention will become more readily apparent from the following detailed description of the invention in which like elements are labeled similarly and in which:

FIG. 1 is an operational diagram for the preparation of an example of a bioavailable cognitive formulation using surface modified lipid-based vesicles with encapsulated actives for better cognitive improvement;

FIG. 2 is a flowchart for the preparation of Surface modified lipid-based vesicles with encapsulated actives for better cognitive improvement for evening times in accordance with the invention;

FIGS. 3a-3d show the results of the Caco-2 permeability assay: 3a) Lutein (LT) permeability, 3b) Sulforaphane (SLP) permeability, 3c) Rutin permeability and 3d) 5-caffeolquinic acid (5-CA) permeability respectively; the permeability is measured in different conditions: with surface modified integrated lipid encapsulation (SMLE) and with/without the presence of the other ingredients as a combination;

FIG. 4 is a schematic representation of the glymphatic system showing operation of the inventive formulation on the glymphatic system; and

FIG. 5 is a graphical representation of the interaction of the inventive formulations to the biological modules for brain health as a result of the invention.

### DETAILED DESCRIPTION OF THE PREFERRED EMBODIMENTS

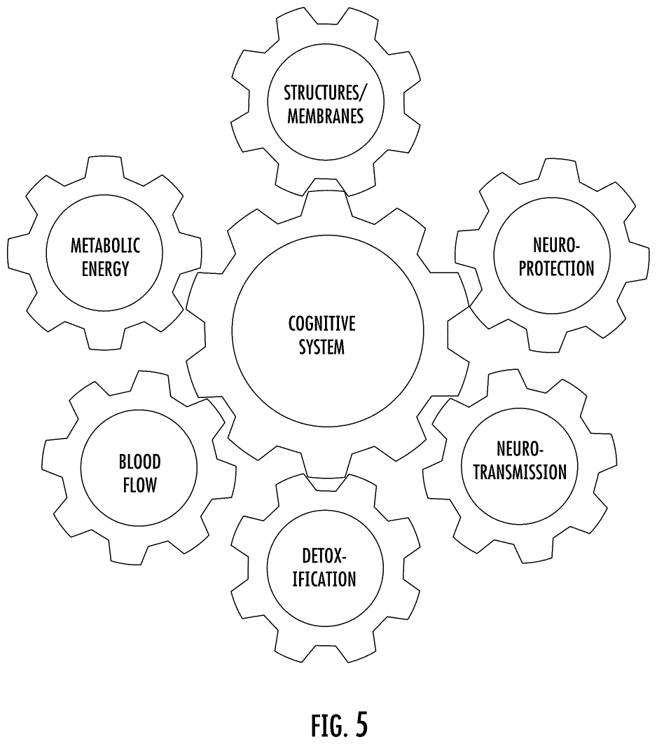

The invention utilizes formulations based on observations on how the brain operates, called the Cognitive system described below (FIG. 5), and the concept that certain nutrients promote brain health at different times during the sleep wake cycle to better take advantage of the glymphatic system. It is believed that a more holistic approach targeting multiple aspects at once could be superior. Especially, a nutritional approach is especially suitable for targeting multiple mechanisms at the same time.

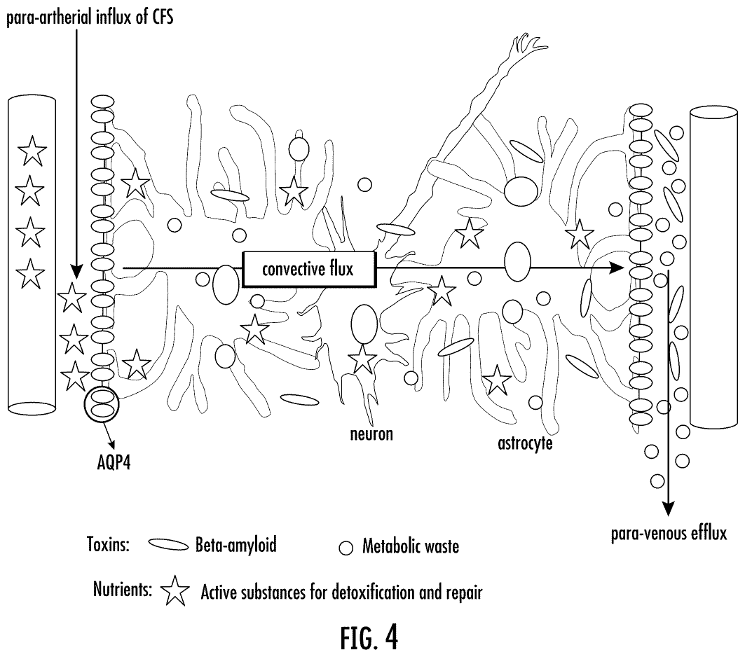

The invention utilizes the operation of the glymphatic system (see FIG. 4) which seems to be active mainly during sleep. Experiments indicate that 90% of the activity happens during the sleep-state. A study with anesthetized mice showed a similar activation of the glymphatic system compared to the sleep-state, indicating that the onset of the glymphatic system is induced by sleep and not the circadian rhythm (Jessen, Finmann Munk, Lundgaard, & Nedergaard, 2015). The key-regulator seems to be norepinephrine. An experiment using an antagonist for the norepinephrine receptor in awake mice induced the glymphatic system comparable to a sleep state (O'Donnell, Zeppenfeld, & McConnell, 2012).

This indicates that the sleep-state is the moment the glymphatic system is clearing the neurotoxic waste products produced during wakefulness. This state also seems to make ingredients better available in the brain. Sleep has been shown to be crucial for mood, cognition, longevity, and a reduced incidence of psychiatric disorders. It is believed for this invention that the glymphatic system plays an important role in this. It has recently been shown that especially during the first hours of sleep the glymphatic system is most active.

The first part of the sleep cycle might also be the moment that the influx of drugs/nutrients by the CSF into the brain and therefore the brain-wide distribution is optimal. This can have serious implications regarding pharmacokinetics when taking nutritional ingredients for brain support.

The invention includes a composition most effective taken before sleep to enhance the bioavailability of the active nutrients in the brain tissue ensuring optimal detoxification.

The inventive formula includes variations based upon a range of about 5 mg rutin and a range of about 5 mg lutein or more in a surface modified integrated lipid encapsulation; SMLE format.

### EXAMPLE

A combined SMLE/non-SMLE formula to be taken before sleep together with a combined SMLE/non-SMLE formula taken during the day with or without an additional lipid-based formula. The timing of the formulations is important. The deepest sleep is normally in the beginning of the sleep cycle. Older people are awake more frequently, sleep less deeply (lack of non-REM 3, shorter non-REM in total) and have a reduced total sleeping time. The sleep quality reduction in older people is negatively impacting the glymphatic flow.

The first part of the sleep cycle might also be the moment that the influx of drugs/nutrients by the CSF into the brain and therefore the brain-wide distribution is optimal. This can have serious implications regarding pharmacokinetics when taking nutritional ingredients for brain support.

Taking advantage of this sleep cycle, a preferred nonlimiting embodiment of the formulation of the invention to be taken at bedtime includes about 15 mg Sulphoraphane or its precursors including glucorapainin for promoting several elements of the Cognitive system: detoxification (activating detox enzymes), neuroprotection (antioxidant effect) and boosting metabolic energy (by mitochondrial biogenesis). The formulation also includes about 30 mg of glucoraphanin; preferably derived from broccoli seed extract may be used instead of or in addition to Sulphoraphane again promoting several elements of the Cognitive system. The formulation further includes about 10 mg of Lutein+Zeaxanthin for neuroprotection. Lutein +Zeaxanthin may be derived from marigolds. The formulation further includes about 330 mg of 5-CQa 5-Caffequinic acid for detoxification. Which may be derived from green coffee. Lastly, the formulation includes Hibiscus extract in the form of about 8.1 mg of gossypetin. Gossypetin contributes to detoxification.

More particularly the formulation may include:

### Evening/Pre-Sleep Formula:

| Surface modified integrated lipid | |
|---|---|
| encapsulation composition (SMLE) | Additional ingredients |
| 1-25 mg rutin and 1-50 mg quercetin | |
| 1-30 mg sulphoraphane | 1-300 mg glucoraphanin |
| 1-25 mg lutein | 1-25 mg zeaxanthin |
| 50-1,000 mg 5-CQa 5-Caffequinic acid | |
| | 1-50 mg gossypetin (hibiscus |
| | extract) |
| 1-500 mg DHA / 1-500 mg EPA | |
| 1-500 mg phosphatidyl choline | |

In a preferred nonlimiting embodiment:

### Evening/Pre-Sleep Formula:

| Surface modified integrated lipid | |
|---|---|
| encapsulation composition (SMLE) | Additional ingredients |
| 10 mg rutin and 10 mg quercetin | |
| 15 mg sulphoraphane | 30 mg glucoraphanin |
| 10 mg lutein | 10 mg zeaxanthin (non meso) |
| 330 mg 5-CQa 5-Caffequinic acid | |
| | 8.1 mg gossypetin (hibiscus extract) |
| 5 mg DHA / 7.5 mg EPA | |
| 50 mg phosphatidyl choline | |

The efficacy of the invention can be further optimized by providing repair nutrients benefiting the structures/membranes and neurotransmission elements (FIG. 5), like DHA (Docosahexaenoic acid) and or EPA (Eicosapentaenoic acid) and UMP (Uridine monophosphate during the day. DHA also lowers triglycerides and increases HDL (good cholesterol) benefiting the blood flow element of the Cognitive system. EPA especially benefits blood flow by addressing natural vasopressor effects when the brain is awake which can otherwise impair blood flow to the brain, and by enhancing the endothelial function.

In another aspect of the invention, compositions are provided for use in the morning to promote the brain's energy metabolism, to stimulate nerve growth factor, to enhance neurotransmission, to support the membranes and structures of the brain, to promote blood flow and to provide neuroprotective nutrients to the brain. These mechanisms for enhancing brain health with a holistic approach are further elucidated in FIG. 5. In this embodiment the composition is formulated utilizing the SMLE delivery system with a composition to be taken during the day, preferably in the morning. The formulation includes about 5 mg to 7.5 mg of the fatty acids DHA/EPA respectively to provide membrane structure, neurotransmission support and blood flow improvement (FIG. 5). The composition is about 15 mg of Erinacine A; preferably in the form of Lion's mane mycelium for the promotion of neurotransmission through the induction of the nerve growth factor. Lastly the composition includes about 56 mg phosphatidyl choline derived from CDP Choline. The resulting Phosphatidyl Choline is also for the promotion of neurotransmission.

The day composition also includes a non-liposome composition to be taken during the day, preferably in the morning is also part of the invention. It may be consumed with the SMLE liposome composition described above. In a preferred non limiting embodiment, the non-liposome composition includes about 200 mcg of Vitamin A derived from retinyl acetate. Vitamin A promotes neuroprotection. The composition includes about 30 mg of Vitamin B-3 (niacin form) to promote blood flow. The composition includes about 0.19 mg of Vitamin B1 (Thiamine) acting as a metabolic energy enhancing element. The composition includes about 0.21 mg of Vitamin B2 (Riboflavin) acting as a metabolic energetic. The composition includes about 5 mcg of Vitamin B7 (Biotin) acting as a metabolic energetic. The composition includes about 0.66 mg of Vitamin B5 (Panthenoic acid) acting as a metabolic energetic.

The day composition also includes about 400 mg of Folate (methyl) to promote neurotransmission. The composition includes about 1 mg of Vitamin B-6 to promote neurotransmission. The composition includes about 3 mcg of Vitamin B-12 (methyl) preferably sourced from methyl cobalamin. Vitamin B-12 also acts as a metabolic energy enhancing element. The composition includes about 80 mg of vitamin C, preferably sourced from sodium ascorbate to promote neuroprotection. The composition includes about 10 mcg of Vitamin D, preferably sourced from cholecalciferol (D3) to promote neuroprotection. The composition includes about 40 mg of Vitamin E (d-alpha tocopherol) to promote neuroprotection.

The day composition also includes about 18 mcg of Vitamin K, preferably sourced from phytomendione, in a non-limiting preferred embodiment, to promote blood flow. The composition includes about 2 mg of zinc, preferably sourced from zinc monomethionine, in a non-limiting preferred embodiment, to promote neurotransmission. The composition includes about 60 mcg of selenium to promote neuroprotection. The composition includes about 20 mcg of Iodine sourced from potassium iodide, in a non-limiting preferred embodiment, to promote neuroprotection. The composition includes about 88 mg of phosphorous to promote neurotransmission. The composition includes about 2 mg of iron, sourced from ferrous lactate, in a non-limiting preferred embodiment, to promote neurotransmission. The composition includes about 300 mcg of copper, sourced from copper gluconate, in a non-limiting preferred embodiment, to promote neurotransmission. The composition may include about 100 mcg of calcium to promote neurotransmission. The composition includes about 50 mg of magnesium, sourced from magnesium glycinate lysinate, in a non-limiting preferred embodiment, to promote neurotransmission.

The composition includes about 410 mcg of manganese, sourced from manganese sulfate, in a non-limiting preferred embodiment, to promote neurotransmission. The composition includes about 100 mcg of chromium, preferably sourced from chromax chromium picolinate, in a non-limiting preferred embodiment, to promote metabolic energy.

The daytime composition also includes about 12.5 mcg of molybdenum, preferably sourced from sodium molybdate, in a non-limiting preferred embodiment, to promote neuroprotection. The composition may also include chloride to promote neurotransmission. The composition includes sodium to promote neurotransmission. The composition may also include potassium to promote neurotransmission. The composition includes about 625 mg of uridine monophosphate which acts as a choline precursor to promote neurotransmission. Lastly, the composition includes about 50 mg of L-Arginine to promote blood flow. As more clearly defined below, the formulation includes:

More particularly the formulation may include:

### Day/Awake Formula:

| surface modified integrated lipid | Additional |
|---|---|
| encapsulation composition (SMLE) | ingredients |
| 1-50 mg rutin and 1-500 mg quercetin | |
| 1-150 mg Lion's Mane mycelium | |
| (containing erinacine A %) | |
| 1-500 mg phosphatidyl choline | |
| 1-500 mg DHA / 1-500 mg EPA | |
| | 1-1,000 mg Uridine |
| | monophosphate |
| | (UMP) |
| | 1-250 mg L-Arginine |
| | 100-800 mcg Vitamin A |
| | 1-50 mg B3 (Niacin) |
| | 0.1-1.0 mg B1 (Thiamin) |
| | 0.1-1.0 mg B2 (Riboflavin) |
| | 1-500 mcg B7 (Biotin) |
| | 0.1-0.9 mg B5 (Panthenoic |
| | acid) |
| | 50-800 mcg folate |
| | 0.1-10 mg B6 |
| | 1-20 mcg B12 |
| | 1-1,000 mg Vitamin C |
| | 1-200 mcg Vitamin D3 |
| | 1-400 mg Vitamin E |
| | 1-50 mcg Vitamin K |
| | 1-15 mg Zinc |
| | 1-120 mcg Selenium |
| | 1-50 mcg Iodine |
| | 1-5 mg Iron |
| | 1-500 mcg Copper |
| | 1-250 mg Magnesium |
| | 1-600 mcg Manganese |
| | 1-200 mcg Chromium |
| | 1-25 mcg Molybdenum |
| | Calcium (ref value 100 mg) |
| | Phosphorous (ref value 88 mg) |
| | Chloride (ref value 156 mg) |
| | Sodium (ref value 125 mg) |
| | Potassium (ref value 188 mg) |

In a preferred nonlimiting embodiment:

### Day/Awake Formula:

| surface modified integrated lipid | |
|---|---|
| encapsulation composition (SMLE) | Additional ingredients |
| 7 mg rutin and 10 mg quercetin | |
| 15 mg Lion's Mane mycelium | |
| (containing erinacine A %) | |
| 56 mg phosphatidyl choline | |
| 5 mg DHA / 7.5 mg EPA | |
| | 625 mg Uridine monophosphate |
| | (UMP) |
| | 50 mg L-Arginine |
| | 200 mcg Vitamin A |
| | 30 mg B3 (Niacin superior form) |
| | 0.19 mg B1 (Thiamin) |
| | 0.21 mg B2 (Riboflavin) |
| | 5 mcg B7 (Biotin) |
| | 0.66 mg B5 (Panthenoic acid) |
| | 400 mcg folate (methyl, |
| | superior form) |
| | 1 mg B6 |
| | 3 mcg B12 (methyl cobalamin) |
| | 80 mg Vitamin C |
| | 10 mcg Vitamin D3 |
| | 40 mg Vitamin E (d-alpha |
| | tocopherol) |
| | 18 mcg Vitamin K (phytomendione) |
| | 2 mg Zinc (Zinc monomethionine) |
| | 60 mcg Selenium |
| | 20 mcg Iodine |
| | 2 mg Iron |
| | 300 mcg Copper |
| | 50 mg Magnesium (Mg glycinate |
| | lysinate, Albion) |
| | 410 mcg Manganese |
| | 100 mcg Chromium (Chromax |
| | chromium picolinate) |
| | 12.5 mcg Molybdenum |
| | Calcium (ref value 100 mg) |
| | Phosphorous (ref value 88 mg) |
| | Chloride (ref value 156 mg) |
| | Sodium (ref value 125 mg) |
| | Potassium (ref value 188 mg) |

The above formulas may be supplemented by taking an additional lipid supplement for providing nutrition for building and repairing the brain's structures and membranes, having the below formulation:

In one nonlimiting embodiment:

### Additional Booster (any Time) Module:

| | |
|---|---|
| Surface Modified Integrated lipid | |
| Encapsulation composition | Additional ingredients |

100-2000 mg DHA Daily
50-1000 mg EPA Daily
1-500 mg phospholipids
1-1000 mg choline

In a preferred nonlimiting embodiment:

### Additional Booster (any Time) Module:

| | |
|---|---|
| Surface Modified Integrated lipid | |
| Encapsulation composition | Additional ingredients |

1190 mg DHA Daily
285 mg EPA Daily
30 mg phospholipids
390 mg choline

The above formulations can be taken separately, but preferably together on a daily basis of the dosages described above. The second and third formulations described above make up a daily supplement which may be taken in the form of a beverage or capsule each morning to nutritionally support the Cognitive system, required for healthy brain function, clear thinking, memory, attention and focus. The formulation features ingredients from the Structures/Membranes, Neurotransmission, and Metabolic Energy and Blood Flow components of the proposed regimen. The formulations will leverage the SMLE system for improved bioavailability.

The first formulation described above is a nightly supplement taken in the form of a beverage or capsule each evening, preferably near bedtime, to protect neurons and nutritionally support the natural cleansing mechanisms in the brain that happen during sleep (glymphatic system target).

The formulations, delivery protocols and their delivery chemistry, discussed below can address the six nutritionally-addressable elements of cognitive health (See FIG. 5):
1. Structures and Membranes
2. Neuroprotection
3. Neurotransmission
4. Detoxification
5. Metabolic Energetics
6. Blood Flow

The Cognitive system shown in FIG. 5 demonstrates that brain health/cognition is complex and multimodal, which is not easy to address with 1 pharmaceutical ingredient. Each element of the system addresses a distinct bio-nutritional pathway relevant for supporting brain function. Each active nutritional ingredient in the formulation(s) ties to one of the six biological modules for brain health identified above.

This, next to the bioavailability, should solve the disadvantage using the pharmacological approach by just focusing on one or two key elements.

To improve and maintain brain health, blood flow is important to bring the right nutrients and oxygen to the brain. Some of these nutrients are active in supporting membrane structure (blood vessel, neuronal cells) in the brain. Other nutrients have a more neuroprotective effect by for example showing antioxidant or anti-inflammatory proprieties. Important for the functioning of a cell is that the energy system works efficient, for example the mitochondrial energy system. Also, crucial in the functioning of the brain is that the neuronal cells can communicate with each other. For that synapse formation is needed which can be promoted by specific nutrients. This leads to an improved neurotransmission.

To improve and maintain brain health, blood flow is important to bring the right nutrients and oxygen to the brain. Some of these nutrients are active in supporting membrane structure (blood vessel, neuronal cells) in the brain. Other nutrients have a more neuroprotective effect by for example showing antioxidant or anti-inflammatory proprieties. Important for the functioning of a cell is that the energy system works efficient, for example the mitochondrial energy system. Also, crucial in the functioning of the brain is that the neuronal cells can communicate with each other. For that synapse formation is needed which can be promoted by specific nutrients. This leads to an improvement in neurotransmission.

Lipid-based encapsulation systems have gained tremendous attention over the years because of their noteworthy properties like high drug-loading capacity, lower toxicity, better bioavailability and biocompatibility, stability in the gastrointestinal tract, controlled release, simpler scale-up, and validation process. Lipid-based encapsulation systems still have some disadvantages like poor drug penetration, limited drug encapsulation, and poor targeting. These disadvantages can be overcome by surface modification as described below. Surface-modified lipid encapsulation systems result in controlled release, enhanced penetration efficiency, and targeted medication delivery.

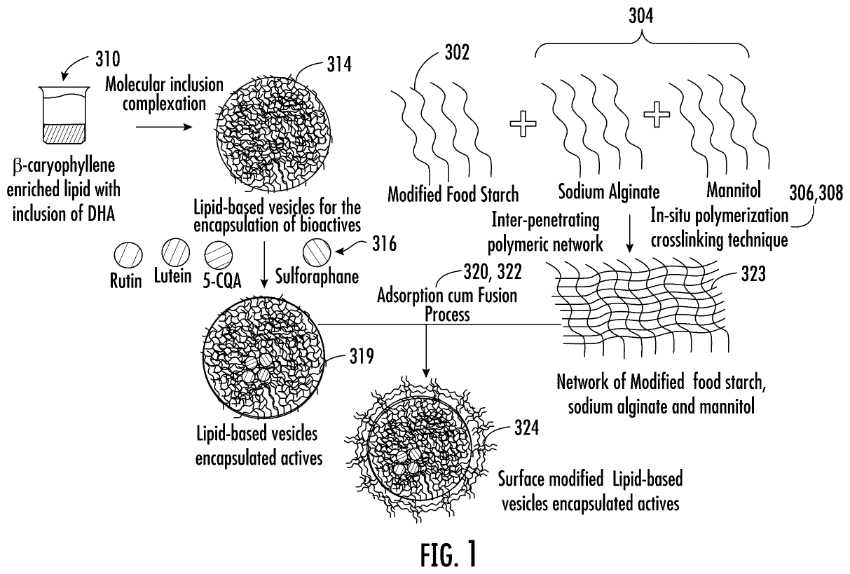

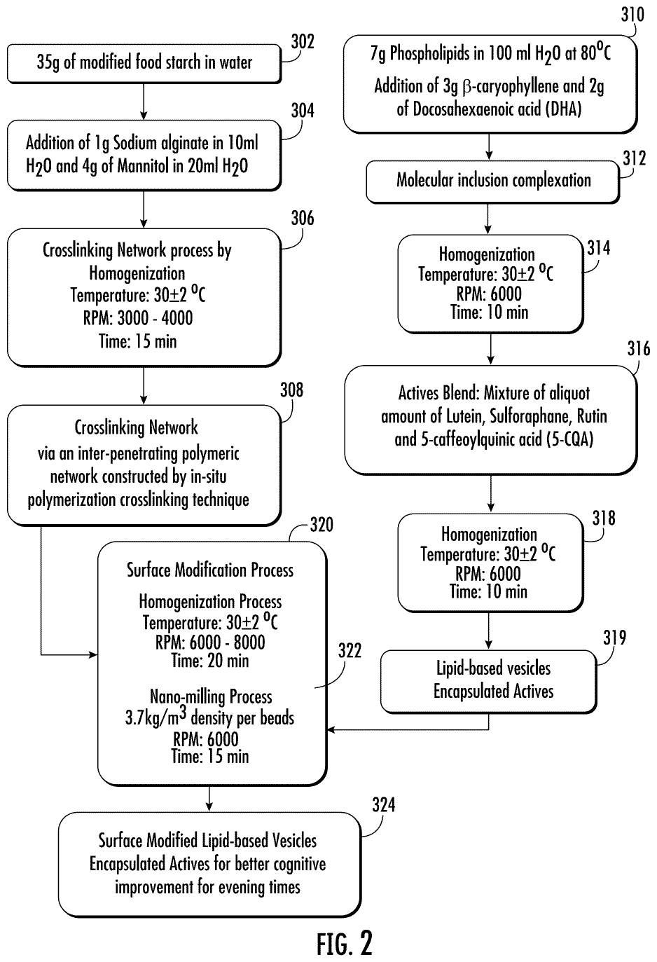

Encapsulating at least part of the formula provides improved results by improving the bioavailability of the active ingredients to the brain. Reference is now made to FIGS. 1 and 2 in which the method of preparation is discussed in detail. In this structure, an innovative crosslinking biopolymer is developed using natural multifunctional polysaccharide biopolymers such as modified food starch, sodium alginate, and mannitol. These are combined via an inter-penetrating polymeric network constructed through in-situ polymerization and crosslinking using hydrogen bonding. This network is applied for surface modification of the lipid-based encapsulated (“SMLE”) formulation using homogenization and nano-milling processes.

Generally, a three-part process is used as a preparation method of Surface-Modified Lipid-Encapsulated Actives (including, but not limited to, Lutein, Sulforaphane, Rutin, Quercetin, 5-Caffeoylquinic Acid (5-CQA)).

### Step 1: Preparation of Crosslinking Biopolymer for Surface Modification

More specifically, in a preferred non limiting embodiment, a crosslinking network is prepared by mixing, in a step 302, about 35 g of modified food starch is mixed with about 100 ml of water, while stirring constantly. In a step 304 about 1 g of sodium alginate is added in about 10 ml of water, followed by the addition of 4 g of mannitol in about 20 ml of water, while stirring constantly. In a step 306, cross linking is promoted by homogenising the mixture at 30±2° C. with stirring at about 3000-4000 RPM for about 15 minutes. In a step 308 crosslinking is continued by constructing an inter penetrating polymeric network constructed using an in-situ polymerization crosslinking technique. The step results in a network of modified food starch, sodium alginate and mannitol 323. Dynamic Light Scattering (DLS) may be used to measure particle size after lipid vesicle preparation or Fourier-transform infrared spectroscopy (FTIR) to confirm crosslinking.

### Step 2: Preparation of Lipid-Based Vesicles for Encapsulation of Bioactives

At the same time, or independently in time, innovative lipid-based vesicles are prepared with an aliquot quantity of phospholipids, β-caryophyllene, and Docosahexaenoic Acid (DHA). β-caryophyllene enriched lipid vesicles are developed with the inclusion of DHA using molecular inclusion complexation technique as follows, β-caryophyllene and DHA were fused together and mixed into the molten phospholipids and homogenized to develop a molecular inclusion complexation. More specifically:

In a step 310, about 7 g of phospholipids are dissolved in 100 ml of water at 80° C. and are subjected to a high shear homogenization at about 6000 RPM. In a step 312, The sheared mixture is added overtime to about 3 g of β-caryophyllene and 2 g of DHA to form β-caryophyllene enriched lipid-based vesicles to form β-caryophyllene enriched lipid-based vesicles with the inclusion of DHA through the molecular inclusion complexation process. In a step 314 the resultant actives mixture is added to the β-caryophyllene enriched lipids-based vesicles with inclusion of DHA and undergo high shear homogenization at about 6000 RPM for 10 minutes to obtain lipid-based vesicles encapsulated actives.

In a step 316, an actives mixture is prepared by adding aliquot amounts of Lutein, Sulforaphane, Rutin, Quercetin, and 5-CQA in 100 ml of water the homogenised lipid-based vesicles solution. In a step 318, the resultant actives mixture is added to the β-caryophyllene enriched lipid-based vesicles with inclusion of DHA and undergo the high shear homogenization with 6000 rpm for 10 min. to obtain lipid-based vesicles encapsulated actives.

### Step 3: Surface Modification of Lipid Vesicles

In a final process the surface of the lipid vesicle is modified to optimize efficacy of the formula. A nano milling process is performed as part of the vesicle preparation process below to obtain the crosslinking network, which is then used for surface modification. The surface modification of lipid vesicles is achieved by adsorption and fusion between the lipid-based vesicles and the crosslinking biopolymer through homogenization and nano-milling processes.

The surface modification of lipid vesicles was achieved by adsorption cum fusion between the lipid-based vesicles and the crosslinking biopolymer through homogenization. The crosslinking polymer network is adsorbed onto and fused into the outer surfaces of the lipid-based vesicles thereby integrating the crosslinking polymeric network with the outer surfaces to modify the outer surfaces of the lipid-based vesiclesIn this design. More specifically, the prepared crosslinking network is slowly added to the mixture of lipid-based vesicles encapsulated actives in a step 320 and then homogenized at 6000-8000 RPM for 20 minutes. Lastly, in a step 322 nano-milling is performed at a density of 3.7 kg/m³ per beads at 6000 RPM for 15 minutes to achieve surface modification.

By following the above strategy, highly bioavailable cognitive formulations via lipid-based encapsulation systems that are able to incorporate hydrophilic, lipophilic, and hydrophobic therapeutic agents. In a preferred embodiment Lutein, Sulforaphane, Rutin and 5-caffeoylquinic acid (5-CQA) were encapsulated in the lipid-based encapsulation system. Based on this design, a formulation can enhance the stability, biocompatibility and drug penetration; particularly across the Blood-Brain-Barrier (BBB) and bioavailability.

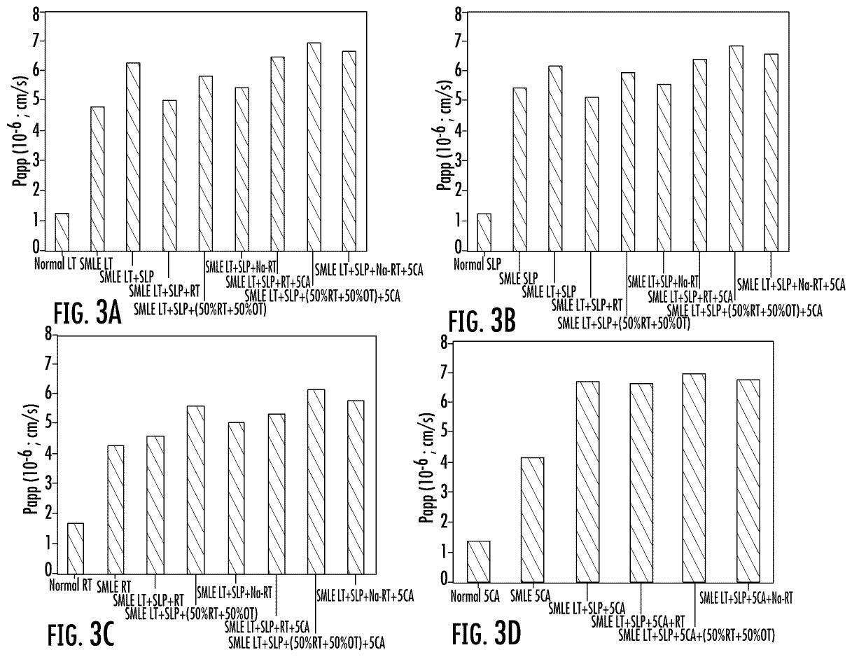

As seen in FIGS. 3a-d the uptake capacity (bioavailability) of the different surface modified integrated encapsulated actives was tested with a Caco-2 permeability assay. The Caco-2 cell-line is a human colorectal adenocarcinoma cell line which is primarily used as a model of the intestinal epithelial barrier. In culture, the cells rapidly differentiate into a heterogeneous mixture of intestinal epithelial cells. This makes the model very suitable to test nutritional ingredient uptake.

Some rationalized strategies as to how permeability values might associate to human oral absorption:
low permeability (0-20% human fraction absorbed (Fa)) is correlated to Papp values of <1-2×10⁻⁶ cm/s
moderate permeability (20-80% Fa) to Papp values of 2-10×10⁻⁶ cm/s
high permeability (80-100% Fa) to Papp >10×10⁻⁶ cm/s

FIGS. 3a-d show the results of the different uptake characteristics of the key nutrients. The permeability is measured in different conditions: with surface modified integrated lipid encapsulation (SMLE) and with/without the presence of the other ingredients as a combination. The surface modified integrated lipid encapsulated formulation shows for all individual ingredients resp: a) Lutein (LT), b) Sulphoraphane (SLP), c) Rutin (RT) and d) 5-caffeoylquinic acid (5-CA) a 3 to 5-fold increase in uptake. This uptake is further enhanced when ingredients are mixed. Except for Rutin which seems to have a negative effect on the uptake of Lutein and Sulphoraphane or its precursors including glucoraphanin (FIGS. 3a and b). The negative effect seems to be neutralized when given in combination with 5-CA.

The conclusion is that each individual ingredient has an improved uptake when encapsulated and even further improved when given in combination with the other ingredients. The permeability values go from low for the individual ingredients without SMLE to high moderate when combined with other ingredients in the SMLE format with permeability increases in the range of 3 to 7-fold. Because SMLEs includes (i) a crosslinking polymeric network, and (ii) lipid-based vesicles having outer surfaces modified with the crosslinking polymeric network, the crosslinking polymer network is adsorbed onto and fused into the outer surfaces of the lipid-based vesicles thereby integrating the crosslinking polymeric network with the outer surfaces to modify the outer surfaces of the lipid-based vesicles, improving ability to cross the blood brain barrier and deliver an effective dosage of the formulation.

Recent studies have shown that CSF and interstitial fluid, the fluid found in spaces around the cells, continuously interchange. There is a convective influx of CSF along the periarterial space (Jessen, Finmann Munk, Lundgaard, & Nedergaard, 2015). The loose fibrous matrix of the perivascular space can be viewed as a low resistance highway for CSF influx. The subsequent transport is facilitated by AQP4 water channels expressed in the astrocytes. The CSF movement into the parenchyma forces convective interstitial fluids towards the perivenous space from where it drains out of the brain toward the cervical lymphatic system. This system of rapid interchange of CSF and interstitial fluid is called the glymphatic system, based on its similarity to the lymphatic system (FIG. 4).

The glymphatic system functions as a clearance pathway for metabolic waste and misfolded proteins. For example, amyloid-β, a protein linked to Alzheimer's disease, is cleared through this system most likely preventing it to form toxic plaques. Growing evidence suggests that glymphatic dysfunction can lead to dementia, worsening the outcomes of stroke and brain trauma and many other neurological disorders (Tithof, et al., 2022). The described formulations, particularly when manufactured as described herein, take advantage of this process.

As a result of the surprising synergies emerging from nutrient combination described above, the above formulations and applications present new and improved formulations and delivery configurations to address brain disease, particularly dementias including Alzheimer's Disease.
1. Using liposomal structures (SMLE) for improving bioavailability for individual nutrients. This results in better delivery of these nutrients is part of the invention.
2. Using liposomal structures (SMLE) for improving bioavailability for nutrient combinations, which are believed to show synergy (see FIG. 3) for the intended health benefit. This results from a novel combination of ingredients packed in a liposome structure with increased bioavailability, a new concept. This can be used for both the morning and evening concept with different ingredients.
3. Using the morning nutrient combinations to positively impact brain health including the structures and membranes, metabolic energy, blood flow and neurotransmission markers is a proposed invention.
4. Using the evening nutrient combinations to detoxify and cleanse the brain during the times that the glymphatic system is active (which may also improve sleep quality) and protect neurons during the sleep cycle is a proposed invention.
5. Providing nutrients to the brain during the day to enhance metabolic energy, neurotransmission, neuroprotection, improve blood flow, and support structures and brain cell membranes. The use of the above formulations before and after sleep as a method of using the product.
6. The above nutritional regimen combines the glymphatic moment of increasing bioavailability with the liposomal opportunity.
7. It is believed that the above formulations and usage regimen can be used to slow down the effects of diseases like mild cognitive impairment, dementias including vascular dementias and Alzheimer's Disease, traumatic brain injuries, migraines, Parkinson's Disease, etc.

The present invention introduces an advanced surface-modified integrated lipid encapsulation system (SMLE) designed to enhance the bioavailability and efficacy of cognitive nutrients. Unlike conventional delivery systems, the lipid matrix in this invention serves a dual role—not only as a delivery vehicle but also as an active component contributing to therapeutic effects. By utilizing bioactive lipids like β-caryophyllene and DHA, the system promotes neuroprotection, anti-inflammatory effects, and improved bioavailability.

Key innovative aspects and benefits include:
1. Dual Functionality of Lipid Matrix: The lipid matrix acts as both a delivery system and an active ingredient, amplifying therapeutic outcomes by leveraging the inherent neuroprotective properties of the lipids used.
2. Enhanced Bioavailability and Targeted Delivery: The SMLE system enhances nutrient penetration across the blood-brain barrier, optimizing cognitive health outcomes.
3. Utilization of the Glymphatic System: The formulation is strategically designed for nighttime administration to align with the glymphatic system's peak activity, improving detoxification and cognitive health.
4. Synergistic Nutrient Combinations: The innovation strategically combines neuroprotective and cognitive-enhancing nutrients, leveraging their synergistic effects for superior therapeutic efficacy.
5. Controlled Release Mechanism: Surface modification techniques facilitate a controlled release profile, ensuring sustained delivery of active ingredients.
6. Scalable Manufacturing Process: A three-step method—comprising crosslinking biopolymer preparation, lipid vesicle formation, and nano-milling surface modification—ensures scalability and industrial viability.

This holistic approach represents a significant advancement in neuro-nutrition, targeting multiple pathways for cognitive health improvement.

It will thus be seen that the objects set forth above, among those made apparent from the preceding description, are efficiently attained and, since certain changes may be made in carrying out the above method and in the construction set forth without departing from the spirit and scope of the invention, it is intended that all matter contained in the above description and shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall there between.

What is claimed is:

1. A method for preparing a surface modified integrated lipid encapsulated structure for a formulation for treating and managing cognitive function and impairment comprising the steps of:
preparing a crosslinking biopolymer for surface modification;
preparing a lipid-based vesicle for encapsulation of a bioactive; and
modifying the surface of the lipid vesicle.

2. The method of claim 1, wherein the formulation includes a rutin and at least one of a quercetin, a sulphoraphane or its precursor glucoraphanin, a lutein, a 5-CQa 5-Caffequinic acid, a Docosahexaenoic Acid (“DHA”), an Eicosapentaenoic acid (“EPA”), or a phosphatidyl choline.

3. The method of claim 1, wherein the formulation includes quercetin and at least one of a rutin, a sulphoraphane or its precursor glucoraphanin, a lutein, a 5-CQa 5-Caffequinic acid, a Docosahexaenoic Acid (“DHA”), an Eicosapentaenoic acid (“EPA”), or a phosphatidyl choline.

4. The method of claim 1, wherein the formulation includes a sulphoraphane or its precursors glucoraphanin, and at least one of a rutin, a quercetin, a lutein, a 5-CQa 5-Caffequinic acid, a Docosahexaenoic Acid (“DHA”), an Eicosapentaenoic acid (“EPA”), or a phosphatidyl choline.

5. The method of claim 1, wherein the formulation includes a lutein and at least one of a rutin, a quercetin, a sulphoraphane or its precursors including glucoraphanin, a 5-CQa 5-Caffequinic acid, a Docosahexaenoic Acid (“DHA”), an Eicosapentaenoic acid (“EPA”), or a phosphatidyl choline.

6. The method of claim 1, wherein the formulation includes a 5-CQa 5-Caffequinic acid and at least one of a rutin, a quercetin, a sulphoraphane or its precursors glucoraphanin, a lutein, a Docosahexaenoic Acid (“DHA”), an Eicosapentaenoic acid (“EPA”), or a phosphatidyl choline.

7. The method of claim 1, wherein the formulation includes a Docosahexaenoic Acid (“DHA”) and at least one of a rutin, a quercetin, a sulphoraphane or its precursor glucoraphanin, a lutein, a 5-CQa 5-Caffequinic acid, an Eicosapentaenoic acid (“EPA”), or a phosphatidyl choline.

8. The method of claim 1, wherein the formulation includes an Eicosapentaenoic acid (“EPA”) and at least one of a rutin, a quercetin, a sulphoraphane or its precursors glucoraphanin, a lutein, a 5-CQa 5-Caffequinic acid, a Docosahexaenoic Acid (“DHA”), or a phosphatidyl choline.

9. A surface modified integrated lipid encapsulation for treating and managing cognitive function and impairment comprising a rutin and at least one of a quercetin, a sulphoraphane or its precursor glucoraphanin, a lutein, a 5-CQa 5-Caffequinic acid, a Docosahexaenoic Acid (“DHA”), an Eicosapentaenoic acid (“EPA”), or a phosphatidyl choline.

10. A surface modified integrated lipid encapsulation for treating and managing cognitive function and impairment comprising a quercetin and at least one of a rutin, a sulphoraphane or its precursor glucoraphanin, a lutein, a 5-CQa 5-Caffequinic acid, a Docosahexaenoic Acid (“DHA”), an Eicosapentaenoic acid (“EPA”), or a phosphatidyl choline.

11. A surface modified integrated lipid encapsulation for treating and managing cognitive function and impairment comprising a sulphoraphane or its precursor glucoraphanin, and at least one of a rutin, a quercetin, a lutein, a 5-CQa 5-Caffequinic acid, a Docosahexaenoic Acid (“DHA”), an Eicosapentaenoic acid (“EPA”), or a phosphatidyl choline.

12. A surface modified integrated lipid encapsulation for treating and managing cognitive function and impairment comprising a lutein and at least one of a rutin, a quercetin, a sulphoraphane or its precursor glucoraphanin, a 5-CQa 5-Caffequinic acid, a Docosahexaenoic Acid (“DHA”), an Eicosapentaenoic acid (“EPA”), or a phosphatidyl choline.

13. A surface modified integrated lipid encapsulation for treating and managing cognitive function and impairment comprising a 5-CQa 5-Caffequinic acid and at least one of a rutin, a quercetin, a sulphoraphane or its precursor glucoraphanin, a lutein, a Docosahexaenoic Acid (“DHA”), an Eicosapentaenoic acid (“EPA”), or a phosphatidyl choline.

14. A surface modified integrated lipid encapsulation for treating and managing cognitive function and impairment comprising a Docosahexaenoic Acid (“DHA”) and at least one of a rutin, quercetin, a sulphoraphane or its precursor glucoraphanin, a lutein, a 5-CQa 5-Caffequinic acid, an Eicosapentaenoic acid (“EPA”), or a phosphatidyl choline.

15. A surface modified integrated lipid encapsulation for treating and managing cognitive function and impairment comprising an Eicosapentaenoic acid (“EPA”) and at least one of a rutin, a quercetin, a sulphoraphane or its precursor glucoraphanin, a lutein, a 5-CQa 5-Caffequinic acid, a Docosahexaenoic Acid (“DHA”), or a phosphatidyl choline.

16. The method of claim 1 wherein the cognitive function and impairment is one of vascular dementia, Alzheimer's Disease, Parkinsons, migraine headaches, and a progression of traumatic brain injuries.

17. The method of claim 1, wherein the managing cognitive function further comprises treating a healthy person to enhance cognitive performance.

18. The surface modified integrated lipid encapsulation of claim 1, wherein the encapsulation is one of a soft gel, a beverage, an edible gel, a tablet; or a capsule.

19. A method for treating and managing cognitive function and impairment comprising the steps of:
administering a surface modified integrated lipid encapsulated structure; the surface modified integrated lipid encapsulated structure having a rutin and at least one of a quercetin, a sulphoraphane or its precursors, a lutein, a 5-CQa 5-Caffequinic acid, Docosahexaenoic Acid (“DHA”), an Eicosapentaenoic acid (“EPA”), or a phosphatidyl choline.

20. A method for treating and managing cognitive function and impairment comprising the steps of:
administering a surface modified integrated lipid encapsulated structure; the surface modified integrated lipid encapsulated structure having a quercetin and at least one of a rutin, a sulphoraphane or its precursors including glucoraphanin, a lutein, a 5-CQa 5-Caffequinic acid, a Docosahexaenoic Acid (“DHA”), an Eicosapentaenoic acid (“EPA”), or a phosphatidyl choline.

21. A method for treating and managing cognitive function and impairment comprising the steps of:
administering a surface modified integrated lipid encapsulated structure; the surface modified integrated lipid encapsulated structure having a lutein and at least one of a quercetin, a sulphoraphane or its precursors including glucoraphanin, a rutin, a 5-CQa 5-Caffequinic acid, a Docosahexaenoic Acid (“DHA”), an Eicosapentaenoic acid (“EPA”), or a phosphatidyl choline.

22. A method for treating and managing cognitive function and impairment comprising the steps of:
administering a surface modified integrated lipid encapsulated structure; the surface modified integrated lipid encapsulated structure having a sulphoraphane or its precursors, glucoraphanin, and at least one of a rutin, a quercetin, a lutein, a 5-CQa 5-Caffequinic acid, a Docosahexaenoic Acid (“DHA”), an Eicosapentaenoic acid (“EPA”), or a phosphatidyl choline.

23. A method for treating and managing cognitive function and impairment comprising the steps of:
administering a surface modified integrated lipid encapsulated structure; the surface modified integrated lipid encapsulated structure having a 5-CQa 5-Caffequinic acid and at least one of a rutin, a quercetin, a sulphoraphane and its precursor glucoraphanin, a lutein, a Docosahexaenoic Acid (“DHA”), an Eicosapentaenoic acid (“EPA”), or a phosphatidyl choline.

24. A method for treating and managing cognitive function and impairment comprising the steps of:
administering a surface modified integrated lipid encapsulated structure; the surface modified integrated lipid encapsulated structure having a Docosahexaenoic Acid (“DHA”) and at least one of a rutin, a quercetin, a sulphoraphane or its precursor glucoraphanin, a lutein, a 5-CQa 5-Caffequinic acid, an Eicosapentaenoic acid (“EPA”), or a phosphatidyl choline.

25. A method for treating and managing cognitive function and impairment comprising the steps of:
administering a surface modified integrated lipid encapsulated structure; the surface modified integrated lipid encapsulated structure having an Eicosapentaenoic acid (“EPA”) and at least one of a rutin, a quercetin, a sulphoraphane or its precursor glucoraphanin, a lutein, a 5-CQa 5-Caffequinic acid, a Docosahexaenoic Acid (“DHA”), or a phosphatidyl choline.

26. A dual function oral delivery system for oral administration to a subject for treating the subject's brain disease, disorder or condition, who is in need of treatment with said oral delivery system, said dual function oral delivery system having the ability to cross the subject's blood-brain-barrier (“BBB”) for (1) providing the subject's brain with neuroprotection, anti-inflammatory protection, and/or anti-inflammatory effects, (2) delivering a bioactive to the subject's brain with improved bioavailability within the subject's brain, and (3) controlling the release of the bioactive from the dual function oral delivery system for sustained treatment of the subject's brain disease, disorder or condition, said dual function oral delivery system comprises:
(a) surface modified lipid encapsulation structures (“SMLEs”) for encapsulation of a bioactive for delivering the bioactive to the subject's brain following oral administration and for controlling the release of the bioactive in the subject's brain to sustain effective treatment of the subject's disease, disorder or condition, wherein the SMLEs includes (i) a crosslinking polymeric network, and (ii) lipid-based vesicles having outer surfaces modified with the crosslinking polymeric network, wherein the crosslinking polymer network is adsorbed onto and fused into the outer surfaces of the lipid-based vesicles thereby integrating the crosslinking polymeric network with the outer surfaces to modify the outer surfaces of the lipid-based vesicles; and
(b) a bioactive encapsulated by the SMLEs in an amount effective to effectively treat the subject's brain disease, disorder or condition; and
wherein the SMLEs, following oral administration, effectively cross the BBB; and
wherein the SMLEs, following penetration across the subject's BBB, provide neuroprotection, anti-inflammatory protection, and/or anti-inflammatory effects to the subject's brain; and
wherein the SMLEs, following penetration across the subject's BBB, improves the bioavailability of the bioactive within the brain; and
wherein the SMLEs, following penetration across the subject's BBB, controls the release of the encapsulated bioactive from the SMLEs into the brain to sustain the effective treatment of the subject's brain disorder, disease or condition; and
whereby, said dual function oral delivery system, following its oral administration to the subject and its penetration across the subject's BBB, provides neuroprotection, anti-inflammatory protection, and/or anti-inflammatory effects to the subject's brain, provides improved bioavailability of the bioactive within the brain, provides controlled release of the bioactive from the dual function oral delivery system, and effectively treats the subject's brain disease, disorder or condition with the bioactive.

27. A formulation for treating and managing cognitive function and impairment comprising a rutin and at least one of a quercetin, a sulphoraphane or its precursor glucoraphanin, a lutein, a 5-CQa 5-Caffequinic acid, a Docosahexaenoic Acid (“DHA”), an Eicosapentaenoic acid (“EPA”), or a phosphatidyl choline.

28. A formulation for treating and managing cognitive function and impairment comprising a quercetin and at least one of a rutin, a sulphoraphane or its precursor glucoraphanin, a lutein, a 5-CQa 5-Caffequinic acid, a Docosahexaenoic Acid (“DHA”), an Eicosapentaenoic acid (“EPA”), or a phosphatidyl choline.

29. A formulation for treating and managing cognitive function and impairment comprising a sulphoraphane or its precursors glucoraphanin, and at least one of a rutin, a quercetin, a lutein, a 5-CQa 5-Caffequinic acid, a Docosahexaenoic Acid (“DHA”), an Eicosapentaenoic acid (“EPA”), or a phosphatidyl choline.

30. A formulation for treating and managing cognitive function and impairment comprising a lutein and at least one of a rutin, a quercetin, a sulphoraphane or its precursors including glucoraphanin, a 5-CQa 5-Caffequinic acid, a Docosahexaenoic Acid (“DHA”), an Eicosapentaenoic acid (“EPA”), or a phosphatidyl choline.

31. A formulation for treating and managing cognitive function and impairment comprising a 5-CQa 5-Caffequinic acid and at least one of a rutin, a quercetin, a sulphoraphane or its precursors glucoraphanin, a lutein, a Docosahexaenoic Acid (“DHA”), an Eicosapentaenoic acid (“EPA”), or a phosphatidyl choline.

32. A formulation for treating and managing cognitive function and impairment comprising a Docosahexaenoic Acid (“DHA”) and at least one of a rutin, a quercetin, a sulphoraphane or its precursor glucoraphanin, a lutein, a 5-CQa 5-Caffequinic acid, an Eicosapentaenoic acid (“EPA”), or a phosphatidyl choline.

33. A formulation for treating and managing cognitive function and impairment comprising formulation includes an Eicosapentaenoic acid (“EPA”) and at least one of a rutin, a quercetin, a sulphoraphane or its precursors glucoraphanin, a lutein, a 5-CQa 5-Caffequinic acid, a Docosahexaenoic Acid (“DHA”), or a phosphatidyl choline.
